(19) United States
(12) Patent Application Publication
(10) Pub. No.: US 2010/0061938 A1
(43) Pub. Date: Mar. 11, 2010

(54) MRI TRACKABLE DRUG DELIVERY PARTICLES, USES AND METHODS THEREOF

(75) Inventors: Sigrid L. Fossheim, Oslo (NO); Esben A. Nilssen, Oslo (NO)

Correspondence Address: BIRCH STEWART KOLASCH & BIRCH, PO BOX 747, FALLS CHURCH, VA, 22040-0747, US

(73) Assignee: EPITARGET AS, Oslo (NO)

(21) Appl. No.: 12/440,914

(22) PCT Filed: Sep. 11, 2007
(86) PCT No.: PCT/NO07/00320

§ 371 Date: Mar. 11, 2009

(30) Foreign Application Priority Data
Sep. 12, 2006 (NO) 20064088

(51) Int. Cl. A61K 49/18

(52) U.S. Cl. 424/9.321

(57) ABSTRACT

The current invention discloses a drug delivery system allowing monitoring of spatial position and drug release, as well as methods and uses thereof. More particularly, the drug delivery system comprises drug carrying particles comprising novel combinations of magnetic resonance imaging contrast agents.

### FIELD OF THE INVENTION

The present invention relates to a drug delivery particle allowing monitoring of spatial position and drug release. More particularly, the invention relates to drug carrying particles comprising magnetic resonance imaging contrast agents, as well as methods and uses thereof.

### BACKGROUND OF THE INVENTION

A serious limitation of traditional medical treatment is lack of specificity, that is, drugs do not target the diseased area specifically, but affect essentially all tissues. This limitation is particularly evident in chemotherapy where all dividing cells are affected imposing limitations on therapy. One strategy to achieve improved drug specificity is incorporation or encapsulation of drugs for example in liposomes, plurogels and polymer particles. To further improve efficiency, ultrasound (US) mediated drug release from such particles has been disclosed in several publications, for a review see Pitt et al. 2004. Other approaches are heat mediated release and light mediated release. All these techniques show promise in laboratory or early preclinical studies, but the clinical, value is yet to be determined. One challenge in this regard is to monitor both accumulation of the drug delivery entity in the diseased area and the extent of drug release.

Magnetic Resonance Imaging (MRI) is an imaging method routinely used in medical diagnostics. The method is based on interactions between radio waves and body tissue water protons in a magnetic field. The signal intensity of a given tissue is dependent on several factors including proton density, spin lattice (T1) and spin spin (T2) relaxation times of tissue water protons. Tissues with shortened T2 will typically appear as an area of low signal intensity on standard T1 or/and T2 (T2*) weighted MR images whilst tissues with shortened T1 will be visualized on standard T1 weighted MR images as an area of high signal intensity.

Contrast agents are used in imaging to increase the signal intensity difference between the area of interest and the background tissue thus enhancing the contrast. In MRI, an increase in signal intensity difference between two tissues is attained by the ability of the contrast agent to selectively shorten the T1 and/or T2 of water protons in a given tissue relative to another. The efficiency of an MRI contrast agent to shorten the T1 and T2 of water protons is defined as the T1 and T2 relaxivity (r1 and r2), respectively.

The higher the relaxivity the more efficient is the agent in shortening the relaxation times of water protons.

Several classes of MRI contrast agents exist, the classification depending on their clinical applications, relaxation and magnetic properties. With respect to magnetic properties, one distinguishes between paramagnetic and superparamagnetic agents. Paramagnetic agents are typically based on the lanthanide metal ions, gadolinium (Gd³⁺), dysprosium (Dy³⁺) and the transition metal ions, manganese (Mn²⁺ and Mn³⁺) and iron (Fe²⁺, Fe³⁺). Due to toxicity, these paramagnetic metal ions need to be administered in the form of stable chelates or other stabilizing entities.

Stabilizing entities may be particulate carriers such as liposomes. Liposomes are spherical colloidal particles consisting of one or more phospholipid bilayers that enclose an aqueous interior. Encapsulation of material in the aqueous interior or incorporation into the phospholipid bilayer provides a means to alter the biodistribution of material and to achieve concentration-time exposure profiles in target tissues that are not readily accomplished with free, i.e. non-liposomal material. Also, the use of sterically stabilised and/or ligand targeted liposome delivery has opened the way for more attractive medical applications, such as medical treatment of tumours and inflammation sites. Some examples of marketed parenteral liposomal drug formulations are: Ambisome®, containing amphotericin B (antifungal agent), Caelyx® containing doxorubicin (chemotherapeutic agent) and DaunoXome® containing daunorubicin (chemotherapeutic agent). Liposomes have also been extensively investigated as carriers for paramagnetic and superparamagnetic materials, but so far no liposomal MRI contrast agents are commercially available.

Liposomes or other particles containing paramagnetic agents shorten the T1 of tissue water protons by so-called dipolar relaxation mechanisms. The latter also contribute to a T2 shortening effect. Another possible contribution to the overall T2 shortening is the susceptibility, also termed T2*, effect of the liposomes (see later paragraph for more details). The ability of paramagnetic liposomes to shorten T1 and/or T2 depends amongst other on the physicochemical properties of both the liposome and paramagnetic agent involved as well as the localization of the latter within the liposome. For instance, in the case of liposome encapsulated Gd chelate the dipolar T1 relaxation effect is mediated by an exchange process of water molecules between the liposome interior and exterior, i.e. bulk water (Barsky et al. 1994). Depending on the physicochemical properties of the liposome and Gd chelate, the dipolar relaxation effect is either in the slow water exchange or fast water exchange regimes. In simplistic terms, the combination of low liposome membrane permeability and encapsulation of Gd agent in sufficiently high amounts will result in an exchange limited relaxation effect yielding an overall low liposome T1 relaxivity. Various in vitro studies have shown how liposome size and composition of the liposome membrane affect the T1 relaxivity of liposome encapsulated Gd agent under conditions of slow water exchange (Tilcock et al. 1989, Fossheim et al. 1999a). Adversely, when the membrane permeability is high enough to relieve any exchange limitations (i.e. fast water exchange regime), the liposomal T1 relaxivity is high and similar to the relaxivity of the free (non-encapsulated) Gd agent (Fossheim et al. 1999a; Fossheim et al. 2000). The same underlying mechanisms apply for dipolar mediated T2 relaxation efficacy of the above systems. However, as long as fast water exchange conditions prevail, liposome encapsulated Gd agent will preferentially act as a T1 agent and increase the signal intensity of a given tissue.

Particulate (e.g. liposomal) paramagnetic agents can also be regarded as a magnetized particle due to the confinement or compartmentalization of a high amount of paramagnetic material within the particle. In such circumstances, long range relaxation mechanisms can develop originating from the magnetic field gradients induced by the difference in magnetic susceptibility between the liposome (containing the agent) and bulk. These long range relaxation mechanisms are not dependent on water exchange and are usually referred to as susceptibility or T2* effects. Susceptibility effects typically decrease the overall T2 and, hence, signal intensity of a given tissue. In order to maximize the susceptibility effects, paramagnetic materials that have a high magnetic susceptibility are used or more preferably superparamagnetic iron oxides are used.

With respect to paramagnetic susceptibility effects, Dy based compounds are usually preferred materials due to a twice as high magnetic susceptibility than Gd based compounds. Indeed, studies have shown the potential of Dy chelates as susceptibility agents per se or present in particles; no interfering T1 effect will occur due to the very poor dipolar relaxation efficacy of Dy³⁺ ions (Fossheim et al, 1997, 1999b).

In functional terms, a liposome encapsulated Gd agent will preferentially function as a T1 agent when factors such as high membrane permeability favour rapid water exchange between liposome interior and exterior. In cases of low membrane permeability and slow water exchange, liposome encapsulated Gd agent will preferentially act as a T2 or susceptibility (T2*) agent. The same conclusions can be drawn for liposomes containing Gd agent incorporated or bound to the inner surface of the liposome membrane. A liposomal Dy agent will only function as a T2 or susceptibility (T2*) agent irrespective of membrane permeability and/or localization within the liposome. Due to the twice as high magnetic suspeptibility, the T2* relaxation efficacy of a given liposomal Dy agent will be higher than that of the analogous liposomal Gd agent in cases of low membrane permeability and slow water exchange, assuming similar or identical liposome loading and size.

Liposomal formulations containing Gd agents are known from the art, while manufacture or use of liposomal Dy chelates have not been reported.

EP1069888B1, incorporated herein by reference in its entirety, discloses a contrast medium for imaging of a physiological parameter, said medium comprising a matrix or membrane material and at least one magnetic resonance contrast generating species, said matrix or membrane material being responsive to a pre-selected physiological parameter and the response is an increased matrix or membrane permeability or chemical or physical breakdown of the matrix or membrane material, to cause the contrast efficacy of said contrast generating species to vary in response to said parameter. '888B1 does not mention coformulation of drugs and contrast agents. Hence, there is no discussion drug release and the need to monitor the spatial position, accumulation and concentration of a drug carrying particle, less the need to monitor the efficiency of drug release. In conclusion, no solution to the current problem is disclosed in '888B1.

Rubesova et al. (2002) describe Gd-labeled liposomes for monitoring liposome-encapsulated chemotherapy. This particle has a high water permeability and displays no water exchange limitations at physiological temperature only making it useful for monitoring spatial position. Hence, the need to concomitantly monitor position, particle concentration and drug release is neither realized nor solved.

Bednarski et al. (1997) report use of liposome encapsulated Gd-DTPA as an MR-detectable model representing pharmaceutical agents. Bednarski et al. use liposomal Gd chelate to track the position of the liposome and will be dependent on a liposome membrane facilitating fast water exchange. Monitoring of drug release is not mentioned and no solution is suggested.

US 2004/0253184 describes a liposome with polymerizable functional groups and a metal chelate for linking of e.g. an MRI contrast agent, as well as antibodies and ligands for in vivo binding of cell surface receptors. The contrast agents are always linked to the liposome membrane and may be e.g. dysprosium or gadolinium based materials. WO 98/44910 discloses a liposome comprising a paramagnetic chelate, e.g. GdDTPA-BMA, and a drug. WO 04/023981 describes so-called envirosensitive liposomes designed to release drugs during specific conditions like high temperature, pH, or acoustic fields. Said liposomes may also comprise a contrast agent, e.g. gadolinium or dysprosium based materials. None of the disclosed inventions are suitable for both monitoring particle position and drug release efficiency.

Also other groups have reported the use of Gd- and Mn loaded liposomes. See Saito et al. (2005), Viglianti et al. (2004). For a review, see Richardson et al. (2005) and Tilcock (1999).

The current inventors have realized the need to monitor the spatial position, accumulation and concentration of a drug carrying particle, as well as the need to is monitor the efficiency of drug release. The present invention is based on the understanding that the above technical problem may be solved by the combined use of a T1 and a susceptibility (T2*) contrast compound per se encapsulated or incorporated in a robust and stable drug delivery particle. Thus, an MRI trackable drug delivery particle allowing monitoring of both spatial coordinates and drug release is disclosed. The invention improves the safety and efficiency of drug delivery from particles, and is particularly useful in ultrasound mediated drug release from particular drug delivery systems.

### DEFINITIONS

The use of singular form may herein mean one or several. Hence, ‘a contrast agent’ means one or several contrast agents, unless specified otherwise.

The terms ‘contrast efficiency’ and ‘relaxation efficiency’ are used interchangeably in the current document.

The term ‘non-physiological parameters’ means physical and chemical parameters not encountered in healthy or diseased mammals. A temperature of 50° C. is an example of a non-physiological parameter.

The term ‘breakdown’ means both chemical and/or physical breakdown. Physical breakdown includes disruption or opening of the matrix or membrane, while chemical breakdown includes dramatic increase in membrane or matrix permeability, e.g. by pore formation. The breakdown may be temporary and/or permanent. In functional terms ‘breakdown’ means release of the carried drug and enhanced overall relaxation enhancement.

The term ‘contrast agent per se’ means herein a MRI contrast compound or a compound with the ability to generate a MR contrast given the right conditions. The term ‘contrast agent’ may be any contrast compound, contrast generating aggregate, contrast agent per se, contrast generating particle or entity.

The term ‘bulk water’ means herein the water compartment exterior to the particle where the majority of water molecules reside.

T2* effect means susceptibility effect that contributes to the overall T2 shortening in compartmentalized systems.

### DETAILED DESCRIPTION OF THE INVENTION

The current invention comprises a trackable particulate material for drug delivery comprising a matrix or membrane material, a drug, and a first and a second magnetic resonance contrast generating species, wherein the relaxation efficiency of said first species is optimal before drug delivery and the relaxation efficiency of said second species is optimal during and/or after drug release. Alternatively, the relaxation efficiency in response to drug release varies in the second species but not necessarily in the first species.

More particularly, the current invention comprises a trackable particulate material for drug delivery comprising a matrix or membrane material, a drug, and a first and a second magnetic resonance contrast generating species, wherein the relaxation efficiency of said first species is optimal in intact matrix or membrane and the relaxation efficiency of said second species is optimal as a result of chemical or physical breakdown of the matrix or membrane material.

More particularly, the current invention comprises a trackable particulate material for drug delivery comprising a matrix or membrane material, a drug, and a first and a second magnetic resonance contrast generating species, wherein the T2 or susceptibility (T2*) relaxation efficiency of said first species is optimal in intact matrix or membrane and the T1 relaxation efficiency of said second species is optimal as a result of chemical or physical breakdown of the matrix or membrane material.

Even more specifically, the current invention comprises a trackable particulate material for drug delivery comprising a matrix or membrane material, a drug, and a T1 and a susceptibility (T2*) magnetic resonance contrast agent (or contrast agent per se), said matrix or membrane material being responsive only to non-physiological parameters and the response is chemical or physical breakdown of the matrix or membrane material, to cause the relaxation efficiency of only the T1 agent to increase.

It is a central feature of the current invention that the T1 agent exhibits low or essentially no T1 relaxation effect before the membrane material or matrix breakdown, while the T2 or susceptibility (T2*) relaxation efficiency of T2* agent is optimal in the intact matrix or membrane material (FIG. 1). In other words, the trackable particulate material as a whole switches from a T2 or T2* agent to a T1 agent as a result of breakdown of the matrix or membrane material and, consequently, coincides with drug release. This feature presupposes that water permeability does not increase without drug release. The T2 (T2*) to T1 contrast switch will typically be visualized as a transition from low signal intensity to high signal intensity on standard T1-weighted or combined T1-weighted and T2 (T2*) weighted MR images (FIG. 2).

The membrane or matrix material may be any material suitable for the current task, e.g. lipids or polymer substances. Moreover, the membrane or matrix material may be an amphiphilic substance capable of forming a liquid crystalline phase, in contact with a liquid selected from the group consisting of water, glycerol, ethylene glycol, propylene glycol and mixtures thereof. The water permeability of the intact matrix or membrane material must, however, impose relaxation exchange limitations, as described above. That is, the permeability, preferably the water permeability, of the membrane or matrix material must possess characteristics not allowing a high level T1 relaxation efficiency of the second species. Typically the membrane permeability will be at a level essentially eliminating any T1 relaxation effects of said second contrast species. Consequently, it is an essential aspect of the present invention that the membrane or matrix material should be non-responsive vis-à-vis both normal and pathological physiological conditions in terms of e.g. temperature, pH, enzyme activity, carbon dioxide tension, oxygen tension, enzyme activity, ion concentration, tissue water diffusion, pressure, tissue, electrical activity. More specifically, the membrane or matrix permeability should not increase in response to normal or pathological physiological conditions in mammals, moreover, the matrix or membrane should not suffer chemical or physical breakdown vis-à-vis said the mentioned conditions. This to ensure that the drug load is not released uncontrolled, but always in response to an extra-corporal stimuli, like e.g. light, ultrasound or non-physiological temperatures.

The membrane or matrix material may form a functionalized cubic gel precursor, functionalized cubic liquid crystalline gel, a dispersion of functionalized cubic gel 30, particles, a functionalized cubic gel particle, gel, precursor, dispersion. It may also form a polymer-based, alginate or chitosan nanoparticle. In a preferred embodiment of the current invention the membrane or matrix material is a phospholipid membrane, forming a liposome. The crystalline gel-to-liquid phase transition temperature (Tc) of the liposome membrane must be higher than normal or pathological physiological temperatures, that is, under no circumstances lower than 42° C.

A liposomal product for parenteral administration demands high chemical and colloidal stability both during storage and use. Additionally, it must be non-toxic and biologically compatible, e.g. isotonic and isohydric. The composition and design of the liposome depend upon the properties and applications of the liposomal product. Charge stabilization of liposomes is achieved by imparting a surface charge to the liposome surface, which is accomplished by employing negatively or positively charged phospholipids. Polymeric coating materials, such as polyethylene glycol (PEG), are also used to prevent particle fusion or aggregation by steric hindrance, thus increasing colloidal stability. Liposomes of high chemical stability are normally obtained by using saturated phospholipids with a gel-to-liquid crystal phase transition temperature (Tc) above 42° C., in practice phospholipids having saturated fatty acid portions with an acyl chain length of 14 carbon atoms or more are used. This is a crucial feature for liposome encapsulated material as the risk of leakage during storage and also in vivo is minimized. For membrane incorporated material, the use of saturated phospholipids is not so critical for minimizing leakage; however the use of saturated phospholipids is preferred to achieve acceptable chemical stability.

The membrane composition chosen will result in liposomes that are physicochemically robust and that retain incorporated or encapsulated material both during extended storage and in vivo. A sterol component could be included to confer suitable physicochemical and biological behavior. The sterol component in the liposomes of the present invention is suitably cholesterol or its derivatives, e.g., ergosterol or cholesterolhemisuccinate, but is preferably cholesterol. The sterol should be present in an amount that enables maximum retention of entrapped or incorporated material, minimizes alterations in physicochemical properties (e.g., liposome size and size distribution) during long-term storage but without negatively affecting the conditions of exchange limitations prior to chemical or physical breakdown of the membrane material. Calcidiol or calcidiol derivates may also be used conveying both structural and therapeutic advantages.

The membrane bilayer of the liposomes of the present invention preferably contains negatively charged and/or neutral phospholipid components in such a combination or mixture that results in an overall Tc above 42° C. Typically, the selected phospholipids will have saturated fatty acid portions with an acyl chain length between 14 and 20 carbon atoms. The neutral phospholipid component of the lipid bilayer is preferably a phosphatidylcholine, most preferably chosen from diarachidoylphosphatidylcholine (DAPC), hydrogenated egg phosphatidylcholine (HEPC), hydrogenated soya phosphatidylcholine (HSPC), distearoylphosphatidylcholine (DSPC), dipalmitoylphosphatidylcholine (DPPC) and dimyristoylphosphatidylcholine (DMPC). The negatively charged phospholipid component of the lipid bilayer may be a phosphatidylglycerol, phosphatidylserine, phosphatidylinositol, phosphatidic acid or phosphatidylethanolamine compound.

Liposomes of the present invention may be prepared by methods that are broadly known in the art (Lasic DD. Preparation of liposomes. In: Lasic DD, ed. Liposomes from physics to applications. Amsterdam, The Netherlands: Elsevier Science Publishers B.V., 1993; 63-107).

The matrix or membrane material of the current invention may comprise photosensitizers, preferably photosensitizers based on the porphyrin skeleton, particularly disulfonated tetraphenylporphine (TPPS2a) or aluminium phthalocyanine (AIPcS2a). These photosensitizers render possible drug release by means of light, acoustic energy or cavitation.

Furthermore the particulate material may comprise an air bubble, e.g. a liposome comprising air bubbles like perfluorobutane, to increase the ultrasound sensitivity. However, an air bubble will typically not be present as this will make particle size exceed 100 nm. Thus, liposomes comprising no air bubbles are preferred. Microbubbles, that is, phospholipid encapsulated air bubbles, are not part of the current invention.

The particulate material may be sensitive to high temperatures, light of defined wavelength, cavitational effects, exogenously generated acoustic energy to induce drug release. High temperatures herein means above normal and pathological physiological levels, typically above 42° C. In a preferred embodiment of the current invention the particulate drug delivery material or matrix or membrane material, e.g. the liposome, is sensitive to acoustic energy, more particularly, ultrasound. An ultrasound sensitive material in the context of drug release means a material responding to ultrasound or acoustic energy by releasing its drug contents. The particular mechanism of release is not relevant, however, relaxation exchange limitations must be suspended during and/or directly after drug release. This typically means disrupting or breaking down the membrane to a degree dramatically increasing the T1 relaxation efficiency of the second contrast species. The ultrasound waves may be of any frequency or amplitude provided that said ultrasound waves induce drug release from the particulate material of the invention. More particularly, it is preferred that the frequencies are below 1.5 MHz, more preferably below 1.1 MHz. In preferred embodiments the frequency is 1 MHz, 500 kHz, 40 kHz or 20 kHz.

The diameter of the particulate material should not exceed 1000 nm. Preferably the diameter is below 1000 nm, more preferably below 250 nm, more preferably below 150 nm, and even more preferably around 100 nm. The current inventors prefer that the size of the material is within the range 50 to 150 nm. Small size is preferred to is maximize the probability of passive accumulation in target tissue due to the Enhanced Permeability and Retention Effect (EPRE) (Maeda et al, 1989).

The drug encapsulated by the current particulate material may be of any suitable chemical or therapeutic type. It is, however, preferred that the drug is hydrophilic or amphiphilic, more preferably hydrophilic. Given that the current invention is related to local release of drugs it is also implied that drugs used should benefit from local release by the current invention. Such drugs are typically anti-inflammatory drugs, antibiotics, anti-bacterial drugs, cardiovascular drugs or anti-cancer drugs. In a preferred embodiment of the current invention the drug is an anti-cancer drug. The particle of the invention may also be designed to incorporate multiple drugs.

As mentioned above, the T1 relaxation efficiency of the second magnetic resonance contrast generating species varies in response to drug release, more specifically, the second contrast species is only MR visible with respect to T1 effect during and/or after drug release. This is possible because drug release, particularly ultrasound induced drug release, will always coincide with relief of the relaxation exchange limitations. Hence, said second species is a T1 contrast agent of any type known to a skilled person, see e.g. EP 1069 88 B1 incorporated herein by reference. Typically, Gd chelates and Mn compounds are used. One or several T1 agent species may be comprised in the drug delivery particle, however, one species is preferred. In a preferred embodiment the T1 contrast agent is a Gd chelate either encapsulated in the aqueous phase of the particulate carrier and/or attached to the inner surface of the particulate carrier membrane. The T1 contrast agent, more particularly the Gd compound, renders qualitative and quantitative monitoring of the drug release process possible.

The above-mentioned T1 agent should be comprised in the aqueous phase of the drug delivery particle, e.g. the liposome, if the drug is hydrophilic. On the other hand, if the drug is amphiphilic or lipophilic, then the T1 agent should be associated with the inner surface of the particulate carrier membrane. Hence, the T1 agent should mimic the solubility properties of the encapsulated drug. In a preferred embodiment, the T1 agent is a hydrophilic compound.

The first magnetic resonance contrast generating species must, as described above, possess a high level relaxation efficiency before drug release. Hence, monitoring of spatial coordinates before drug release is made possible. In this way sufficient particle accumulation in the diseased volume, e.g. tumour, may be ensured before induction of drug release. Hence, the first magnetic resonance contrast generating species is a T2* (susceptibility) agent per se of any suitable type known to a person skilled in the art, see e.g. EP 1069 88 B1. One or several T2* agent species may be comprised in the drug delivery particle, however one species is preferred. Typically, Dy or iron oxides compounds are employed. In a preferred embodiment the T2* agent per se is a Dy chelate. The T2* agent may reside in the membrane or matrix material, on the exterior or interior surface of said material, or in aqueous phase of the particulate material. In a preferred embodiment of the current invention both the T1 and T2* agents are hydrophilic compounds in the aqueous phase of a particle. However, in order to improve the drug carrying capacity of said material, the T2* agent may be positioned on the outside surface.

In functional terms, the susceptibility (T2*) effect of the first magnetic resonance contrast generating species present in intact particles is exploited to monitor extent of particle accumulation in the diseased volume, whilst the T1 effect of the second magnetic resonance contrast generating species induced as a result of membrane or matrix breakdown is exploited for monitoring of drug release.

Another aspect of the current invention is use of the particulate material described supra for the manufacture of a particulate drug delivery system for treating cancer, cardiovascular disease, immunological and inflammatory disease.

A further aspect of the present invention is use of the particulate material of the invention for monitoring spatial position of said material before drug release and efficiency of drug release.

The present invention also comprises use of a particulate material comprising a matrix or membrane material, a drug, and at least two magnetic resonance contrast generating species, said matrix or membrane material being responsive to any pre-selected physiological or non-physiological parameter and the response is an increased matrix or membrane permeability or chemical or physical breakdown of the matrix or membrane material, to cause the contrast efficacy of said contrast generating species to vary in response to said parameter for the manufacture of a particulate drug delivery system for treating cancer, cardiovascular disease, immunological and inflammatory disease. Preferably, the at least two magnetic resonance contrast generating species are one T1 and one T2* agent per se as described above.

Also, the present invention comprises use of a particulate material comprising a matrix or membrane material, a drug, and T1 and a T2* magnetic resonance contrast agent, said matrix or membrane material being responsive to a pre-selected physiological parameter and the response is chemical or physical breakdown of the matrix or membrane material, to cause the relaxation efficacy of said contrast generating species to vary in response to said parameter for the manufacture of a particulate drug delivery system for treating cancer, cardiovascular disease, immunological and inflammatory disease.

Furthermore, the present invention comprises use of a particulate material comprising a matrix or membrane material, a drug, and T1 and a T2* magnetic resonance contrast agent, said matrix or membrane material being responsive to a non-physiological parameter and the response is chemical or physical breakdown of the matrix or membrane material, to cause the relaxation efficacy of said contrast generating species to vary in response to said parameter for the manufacture of a particulate drug delivery system for treating cancer, cardiovascular disease, immunological and inflammatory disease.

Furthermore, the present invention comprises use of a particulate material comprising a matrix or membrane material, a drug, and at least two magnetic resonance contrast generating species, said matrix or membrane material being responsive to a pre-selected physiological parameter and the response is chemical or physical breakdown of the matrix or membrane material, to cause the relaxation efficacy of said contrast generating species to vary in response to said parameter for the manufacture of a to particulate drug delivery system for treating cancer, cardiovascular disease, immunological and inflammatory disease.

In all forms of use the drug delivery or drug activation is preferably induced by ultrasound. Hence, ultrasound exposure is the preferred mode of administration.

The current invention further comprises a method of monitoring particle position and drug release in a mammal comprising the steps of administering parenterally to said mammal the particulate drug delivery material of the present invention; generating T2 or T2* weighted image data of at least part of said body in which said material is present; and generating therefrom a signal indicative of the level of accumulation of said material; generating T1 weighted image data prior to inducing drug release; generating thereafter T1 weighted image data of at least part of said body in which said material is present; and generating therefrom a signal indicative of the level of drug release. The ‘level of drug release’ indicates the quantitative and/or qualitative level of release. Precontrast images are usually generated before contrast agents are administered to the patient.

### BRIEF DESCRIPTION OF DRAWINGS

FIG. 1. Relaxation mechanisms of particulate carrier (e.g. liposome) containing both a T1 and T2 or T2* (susceptibility) agent per se prior to (a) and, after (b) membrane or matrix breakdown. The two agents are different chemical entities.

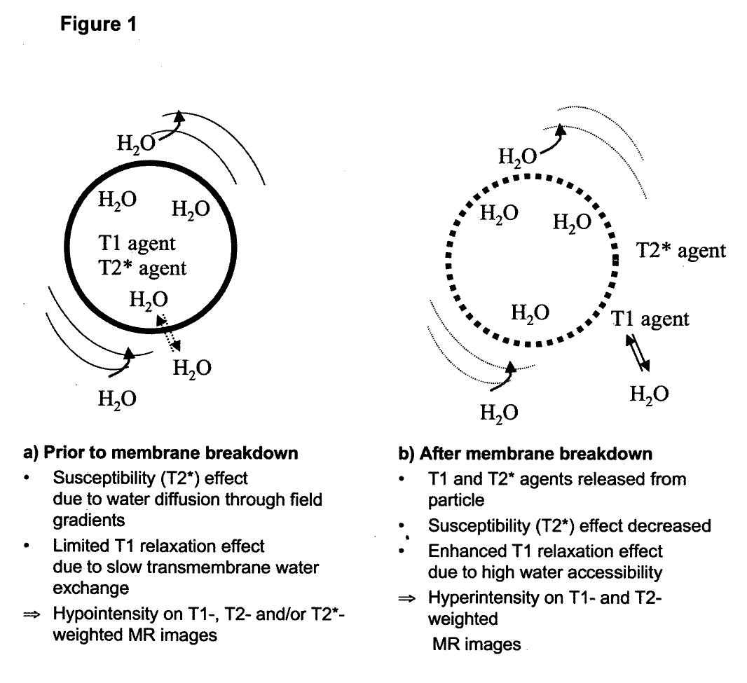

FIG. 2. Schematic and simplified representation of a particulate T2(T2*)−T1 contrast switch where the signal intensity on standard MR images is increased as a result of membrane or matrix breakdown of the particulate carrier.

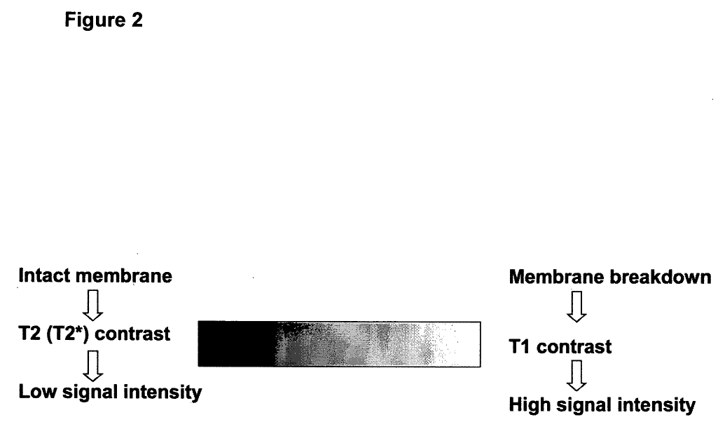

FIG. 3. T2-w SE images of gel phantoms prior to and after ultrasound (US) treatment and surfactant treatment of samples.

### EXAMPLES

The following examples are meant to illustrate how to make and use the invention. They are not intended to limit the scope of the invention in any manner or to any degree.

### Example 1

### Preparation and Characterization of Liposome Containing a Gd Chelate and a Dy Chelate (“Paramagnetic Liposomes”)

DSPC and DSPE-PEG 2000 were purchased from Genzyme Pharmaceuticals (Liestal, Switzerland). Chloroform, methanol, calcein, HEPES, sodium azide and sucrose were all obtained from Sigma Aldrich. GdDTPA-BMA (Omniscan®) and DyDTPA-BMA (Sprodiamide) were kindly supplied by GE Healthcare and Rikshospitalet-Radiumhospitalet, respectively, both Oslo, Norway.

DSPC/DSPE-PEG 2000 (mole %; 92:8) liposomes were prepared by the thin film hydration method. The phospholipids were dissolved in a chloroform/methanol mixture (volume ratio; 10:1) and the organic solution was evaporated to dryness under reduced pressure. Liposomes were formed by hydrating the lipid film with a pre-heated (65 deg C) 10 mL aqueous solution containing GdDTPA-BMA chelate (150 mM), DyDTPA-BMA chelate (150 mM) and 4 mM HEPES (pH 7.4). The resulting liposome dispersion at a nominal phospholipid concentration of 30 mg/mL was allowed to swell during rotation at 65 deg C. for 120 minutes. The liposome dispersion was subjected to three freeze-thaw cycles and extruded at 65 deg C. through polycarbonate filters of various pore diameters to achieve a liposome size below 110 nm. Untrapped materials were removed by exhaustive dialysis (MWCO 100 000 D) against isosmotic sucrose/10 mM HEPES (pH 7.4) solution containing 0.02% w/v sodium azide (used as bacteriostatic agent). Completion of dialysis was determined by relaxometry of last dialysis water.

The dialysed Liposome dispersion was characterised with respect to key physicochemical properties by use of well-established methodology.

The average particle size (intensity weighted) and size distribution were determined by photon correlation spectroscopy (PCS) at a scattering angle of 173° and 25 deg C. (Nanosizer, Malvern Instruments, Malvern, UK). Prior to sample measurements the instrument was tested by running a latex standard (60 nm). For the PCS measurements, 10 μL of liposome dispersion was diluted with 2 mL filtered isosmotic sucrose solution containing 10 mM HEPES (pH 7.4) and 0.02% (w/v) sodium azide. Duplicates were analysed. Osmolality was determined on non-diluted liposome dispersions by freezing point depression analysis (Fiske 210 Osmometer, Advanced Instruments, MA, US). Prior to sample measurements, a reference sample with an osmolality of 290 mosmol/kg was measured; if not within specifications, a three step calibration was performed. Duplicates were analysed. For Gd and Dy content analysis, the liposome dispersion was diluted with 0.2% (w/v) Triton X-100 prior to analysis by inductively coupled plasma atomic emission spectroscopic (ICP-AES) using a Perkin Elmer Optima 3300 DV instrument.

The intensity-weighted diameter of the dialysed liposomes was 108 nm. The osmolality of the dialysed liposome dispersion was 550 mosmol/kg water. The effective concentration of Gd and Dy in the dialysed liposome dispersion was 6.5 mM.

### Example 2

### Preparation and Characterization of Liposome Containing a Gd Chelate, a Dy Chelate and the Drug Marker Calcein (“Paramagnetic Liposomes Containing Calcein”)

DSPC/DSPE-PEG 2000 (mole %; 92:8) liposomes were prepared analogously to Example 1, except that the aqueous solution used for lipid film hydration also contained 20 mM of the fluorescent dye calcein. The dialysed liposome dispersion was characterised with respect to key physicochemical properties as described in Example 1.

The intensity-weighted liposome size was 88 nm. The osmolality of the dialysed liposome dispersion was 640 mosmol/kg water. The effective concentration of Gd and Dy in the dialysed liposome dispersion was 5.2 mM.

### Example 3

### Ultrasound Treatment of Liposome Samples

For the ultrasound experiments, the dialysed liposome dispersions (from Examples 1 and 2) were further diluted with isosmotic sucrose/10 mM HEPES (pH 7.4)/5 mM EDTA solution containing 0.02% w/v sodium azide. EDTA was supplied as the disodium and diydrate salt from Sigma Aldrich.

The ultrasound experiments were performed with a ‘Vibra-Cell’ 40 kHz ultrasonic processor, VC754, with a 1.9 cm diameter transducer, purchased from Sonics and to Materials, Inc. (CT, US). Ultrasound with a 20% amplitude was applied for 4 minutes to the diluted liposome dispersions (dispensed in plastic flask). The plastic flasks containing liposomes were during ultrasound exposure placed in a water/ice bath to minimise any ultrasound mediated heating effect. The sample temperature rose from 10 deg C. prior to ultrasound treatment to 20 deg C. after completed treatment.

### Example 4

### Release Quantification

The extent of ultrasound mediated release could be estimated for the paramagnetic liposomes containing calcein.

The release assessment of calcein is based on the following well-established methodology: Intact liposomes containing calcein will display low fluorescence intensity due to self-quenching caused by the high intraliposomal concentration of calcein (here 20 mM). Ultrasound mediated release of calcein to the extraliposomal phase can be appreciated by a marked increase in fluorescence intensity due to a reduced quenching effect. The following equation is used for release quantification:

$$\% release = \frac{( {F}_{u} - {F}_{b} )}{( {F}_{T} - {F}_{b} )} \times 100$$

Where F_{b} and Fᵤ are, respectively, the fluorescence intensities of the liposomal calcein sample before and after ultrasound application. F_{T} is the fluorescence intensity of the liposomal calcein sample after solubilisation with surfactant, here Triton X-100 (Sigma Aldrich). Studies have shown that the addition of 10 μl Triton X-100 (10%) solution per 1 ml of diluted liposome dispersion followed by 5 minutes heating at 64 deg C. results in about 100% release of calcein.

The fluorescence measurements were carried out with a luminescence spectrometer model LS50B (Perkin Elmer, Norwalk, Conn.) equipped with a photomultiplier tube R3896 (Hamamatsu, Japan). Fluorescence measurements are well known to a person skilled in the art.

For the paramagnetic liposomes containing calcein subjected to 4 minutes of 40 kHz ultrasound at 20% amplitude (Example 3), the release extent of calcein was about 20%. Due to the similar hydrophilicity and molecular weight, the release extent of the investigated Gd and Dy DTPA-BMA chelates can be considered similar for the given liposome formulation (see Example 6).

The investigated liposome formulations have a gel-to-liquid crystalline phase temperature (Tc) above 55 deg C. Since the temperature exposure during ultrasound is was at the most 20 deg C. (Example 3), it can also be concluded that the observed liposome release was not heating related but directly attributed to an ultrasonic effect (most likely cavitation).

### Example 5

### Preparation of Gel Phantoms for MR Imaging

Two gel phantoms were prepared. One gel phantom contained liposome and control samples that have not been exposed to any ultrasound treatment, the second contained samples that first have been exposed to ultrasound followed by surfactant treatment.

MR imaging was performed in three steps: 1) on the first gel phantom containing non-ultrasound treated samples, 2) on the second gel phantom containing ultrasound treated samples and, 3) after addition of surfactant to the inserts containing ultrasound treated samples from 2). Please note, the inserts added with Triton X-100 could not for practical reasons be heated; hence the surfactant treatment will most likely not result in 100% release. Consult Example 6 for more detailed information on the MR experiments

Irrespective of type of phantom, each phantom consisted of 8 glass inserts (i.d. 8 mm) placed in an equidistant manner in one circular glass container. The latter was filled with 1% (w/v) agar (Sigma Aldrich) gel containing 0.25 mM GdDTPA (Magnevist®) giving a T1 of 700 ms at 1.5 T and ambient temperature. Two inserts were dispensed with a 2 mL diluted dispersion of paramagnetic liposomes at an effective concentration of 0.8 and 1.6 mM of both Gd and Dy (present as DTPA-BMA chelate). Two other inserts were dispensed with a 2 mL diluted dispersion of paramagnetic liposomes containing calcein at an effective concentration of 0.65 and 1.3 mM of both Gd and Dy (present as DTPA-BMA chelate). All four liposome samples contained as matrix an isosmotic sucrose solution containing 1.5% PVP (Sigma Aldrich) and 10 mM HEPES (pH 7.4). The other three glass inserts were dispensed with isosmotic sucrose/10 mM HEPES (pH 7.4)/1.5% PVP solutions containing 1 mM GdDTPA-BMA, 1 mM DyDTPA-BMA and a mixture of 1 mM DyDTPA-BMA and 1 mM GdDTPA-BMA, respectively. The remaining glass insert was filled with isosmotic sucrose/10 mM HEPES (pH 7.4) solution containing 1.5% PVP.

### Example 6

### MR Imaging Pre/Post Ultrasound and Surfactant Exposure

T1-weighted (w) and T2-w spin echo (SE) images of the phantoms containing untreated samples, ultrasound treated samples and ultrasound/surfactant treated samples were acquired at 1.5 T using a Siemens Sonata scanner equipped with a 8-channel phased array head coil. T1 relaxation times were estimated using an inversion recovery prepared gradient echo sequence where the inversion time (TI) was varied between 100 ms and 3000 ms. T2 relaxation times were estimated using multi-echo SE sequences where the echo time (TE) was varied over the appropriate range of T2 values of the samples.

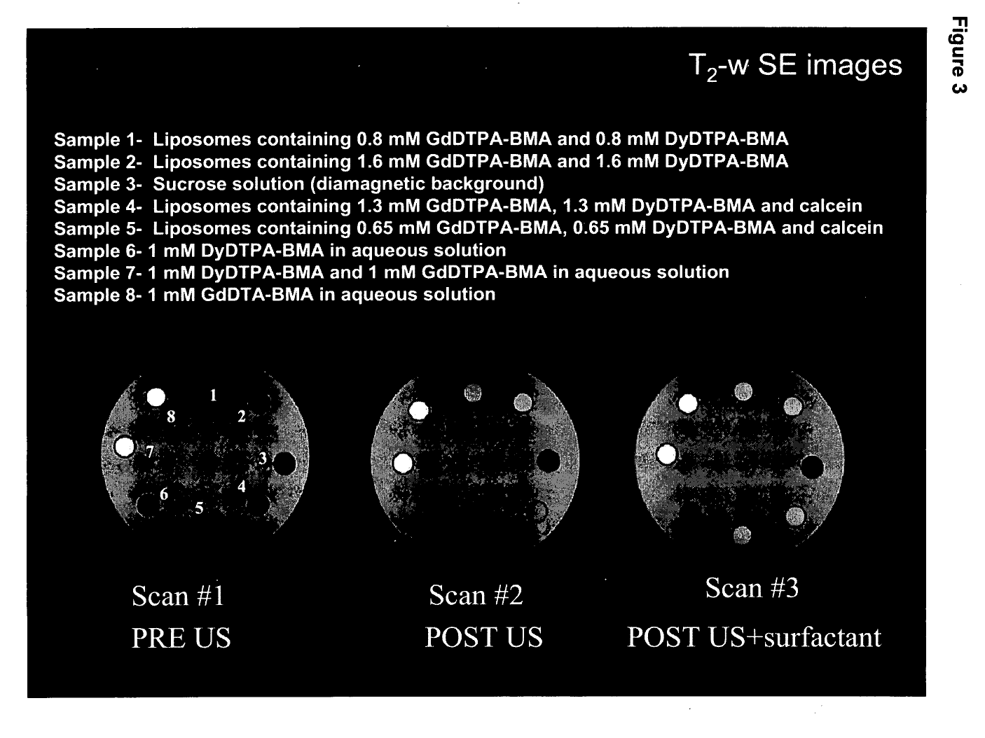

FIG. 3 shows three sets of T2-w SE images, respectively, of the gel phantoms prior to and after ultrasound treatment and surfactant treatment of liposome and control samples. Table 1 shows the T1 relaxation times of the various liposome and control samples prior to and after ultrasound treatment, and after surfactant treatment.

On the T2-w images, it was shown that the paramagnetic liposomes (with or without calcein) were hypointense prior to ultrasound treatment. The signal intensity increased markedly after 4 minutes of ultrasound therapy and increased further after solubilisation with surfactant (Triton X-100). The two control samples containing non-liposomal GdDTPA-BMA (1 mM) and an aqueous mixture of GdDTPA-BMA and Dy-DTPA-BMA (both at 1 mM) were hyperintense prior and after ultrasound treatment.

The control samples containing 1 mM of non-liposomal DyDTPA-BMA was hypointense prior and after ultrasound treatment. The same observations could be drawn for the T1-w SE images (data not shown) with increasingly hyperintense liposome samples as a result of ultrasound treatment and consequent surfactant treatment. These conclusions were supported by the markedly shortened T1 relaxation times for the liposome samples, whilst the T1 relaxation times of the control samples were, within experimental errors, not affected by ultrasound and/or surfactant treatment.

TABLE 1
T1 relaxation times (@ 1.5 T) of liposome/control samples prior to and
after ultrasound (US) treatment, as well as after surfactant treatment (see FIG.
3 for sample legends).

| | | | | Sample 4 | Sample 5 | | | |
|---|---|---|---|---|---|---|---|---|
| | | | | Liposomes | Liposomes | | | Sample 8 |
| | Sample 1 | Sample 2 | Sample 3 | 1.3 mM | 0.65 mM | Sample 6 | Sample 7 | Control |
| | Liposomes | Liposomes | Control | Gd/Dy | Gd/Dy | Control | Control | 1 mM |
| | 0.8 mM | 1.6 mM | Sucrose | and | and | 1 mM Dy | 1 mM Gd | Gd/Dy |
| | Gd/Dy | Gd/Dy | solution | calcein | calcein | solution | solution | solution |
| PRE US | | | | | | | | |
| T1 (ms) | 1063 | 883 | 986 | 948 | 965 | 923 | 137 | 144 |
| POST US | | | | | | | | |
| T1 (ms) | 257 | 138 | 1108 | 351 | 564 | 937 | 139 | 143 |
| POST US+ | | | | | | | | |
| surfactant | | | | | | | | |
| T1 (ms) | 194 | 119 | 1044 | 149 | 242 | 978 | 146 | 143 |

Note:
The Dy and Gd concentrations in the Table imply concentrations of DyDTPA-BMA and Gd-DTPA-BMA.

Also, the concentration corrected paramagnetic T1 relaxation rates of the liposome samples (corrected to 1 mM Gd) after surfactant treatment were somewhat lower than the relevant control samples containing 1 mM GdTDPA-BMA (alone or in mixture) suggesting that the surfactant treatment of the inserts prior to MR imaging (involving no heat) did not result in 100% release but a release of minimum 75%. Note that the release extent estimated in Example 4 is based on surfactant and heated treated liposome samples.

Assuming that ultrasound treatment disrupts a given proportion of liposomes with full release of the encapsulated material, it can be assumed that the observed T1 relaxation time or rate after ultrasound treatment reflects the paramagnetic contribution of both intact liposomes (not disrupted) and released GdDTPA-BMA from the liposomes (the T1 relaxation contribution of DyDTPA-BMA can be neglected here). With this assumption and taking into consideration the T1 relaxation data for both liposome and control samples (Table 1), an approximate 20% release of GdDTPA-BMA (and herein DyDTPA-BMA) could be estimated for the paramagnetic liposomes containing calcein after 4 minutes of 40 kHz ultrasound at 20% amplitude; this result is in very good agreement with the release extent estimated from fluorescence data, also about 20% (See example 4). Analogously, the ultrasound mediated release extent based on T1 relaxation data was estimated to about 60% for the paramagnetic liposomes. The markedly different release extent of the two investigated liposome formulations was most likely due to the different extent of cavitation generated during the ultrasound treatment. The difference in liposome size of about 20 nm cannot explain the markedly different release extent.

Based on the above data and considerations, it can be concluded that the investigated liposome formulations containing both GdDTPA-BMA and DyDTPA-BMA function as a T2(T2*)−T1 switch in simple aqueous media. It is believed that the T2 (T2*)−T1 switch capabilities will be improved in more biological relevant media due to an increased degree of compartmentalization that favour susceptibility effects.

Having now fully described the present invention in some detail by way of illustration and example for purpose of clarity of understanding, it will be obvious to one of ordinary skill in the art that same can be performed by modifying or changing the invention by with a wide and equivalent range of conditions, formulations and other parameters thereof, and that such modifications or changes are intended to be encompassed within the scope of the appended claims.

### REFERENCES

Barsky et al. Theory of paramagnetic contrast agents in liposome systems. Magn Reson Med. 1994; 24:1-13.
Bednarski et al. In vivo target-specific delivery of macromolecular agents with MR-guided focused ultrasound. Radiology. 1997; 204: 263-268.
EP1069888B1 Fossheim et al. Use of particulate contrast agents in diagnostic imaging for studying physiological parameters.
Fossheim et al. Low molecular weight lanthanide contrast agents: in vitro studies of mechanisms of action. J Magn Reson Imaging. 1997; 7:251-7.
Fossheim et al. Paramagnetic liposomes as MRI contrast agents: influence of liposomal physicochemical properties on the in vitro relaxivity. Magn Reson Imaging. 1999a; 17:83-9.
Fossheim et al. Thermosensitive paramagnetic liposomes for temperature control during MRI guided hyperthermia in vitro feasibility studies. Acad Radial. 2000; 12:1107-1115.
Fossheim et al. Investigation of lanthanide-based starch particles as a model system for liver contrast agents. J Magn Reson Imaging. 1999b; 9:295-303.
Lasic DD. Preparation of liposomes. In: Lasic DD, ed. Liposomes from physics to applications. Amsterdam, The Netherlands: Elsevier Science Publishers B.V., 1993; 63-107).
Maeda H, Matsumura Y. Tumoritropic and lymphotropic principles of macromolecular drugs. Crit Rev Ther Drug Carrier Syst, 1989; 6: 193-210.
Pitt et al. Ultrasonic drug delivery- a general review. Expert Opin Drug Deliv. 2004 November; 1:37-56.
Richardson et al. Pharmaceutical applications of magnetic resonance imaging (MRI). Adv Drug Deliv Rev. 2005; 57:1191-209.
Rubesova et al. Gd-labeled liposomes for monitoring liposome-encapsulated chemotherapy: quantification of regional uptake in tumor and effect on drug delivery. Acad Radiol. 2002; 9 Suppl 2:S525-7.
Saito et al. Gadolinium-loaded liposomes allow for real-time magnetic resonance imaging of convection-enhanced delivery in the primate brain. Exp Neurol. 2005; 196:381-9.
Tilcock et al. Liposomal Gd-DTPA: preparation and characterization of relaxivity. Radiology. 1989; 171:77-80.
Tilcock et al. Delivery of contrast agents for magnetic resonance imaging, computed tomography, nuclear medicine and ultrasound. Adv Drug Deliv Rev. 1999; 37:33-51.
Viglianti et al. In vivo monitoring of tissue pharmacokintetics of liposome/drug using MRI: Illustration of targeted delivery. Magnetic Resonance in Medicine, 2004; 51:1153-1162.
US 2004/0253184
WO 98/44910
WO 04/023981

1-11. (canceled)

12. A trackable particulate material for drug delivery comprising a matrix or membrane material, a drug, and a T1 and T2* magnetic resonance contrast agent, the T1 agent having limited access to bulk water, and said matrix or membrane material being responsive only to non-physiological parameters and the response is chemical or physical breakdown of the matrix or membrane material, to cause the T1 relaxation efficiency of the T1 agent to increase, wherein the T1 agent is a gadolinium and/or manganese compound and the T2* agent is a dysprosium and/or iron oxide compound.

13. The particulate material of claim 12, wherein the matrix or membrane material is a phospholipid membrane.

14. The particulate material of claim 12, wherein said matrix or membrane material comprises a liposome.

15. The particulate material according to claim 12, wherein said material comprises two magnetic resonance contrast generating species.

16. The particulate material of claim 12, wherein the T1 agent is a gadolinium compound and the T2* agent is a dysprosium compound.

17. The particulate material of claim 12, wherein said material is for medical use.

18. The particulate material of claim 12, wherein T1 and T2* agents are a hydrophilic compound comprised in the aqueous phase of the particle.

19. A method of treating cancer, cardiovascular disease, immunological or inflammatory disease comprising administering the particulate material of claim 12 to a patient in need thereof.

20. The particulate material of claim 13, wherein said phospholipid membrane comprises a liposome.
